(19) United States
(12) Patent Application Publication
(10) Pub. No.: US 2020/0216427 A1
(43) Pub. Date: Jul. 9, 2020

(54) SOLID STATE FORMS OF ENTRECTINIB

(71) Applicant: TEVA PHARMACEUTICALS INTERNATIONAL GMBH, Jona (CH)

(72) Inventor: Sanja Matecic Musanic, Zagreb (HR)

(21) Appl. No.: 16/737,184

(22) Filed: Jan. 8, 2020

Related U.S. Application Data
(60) Provisional application No. 62/789,577, filed on Jan. 8, 2019.

(51) Int. Cl. C07D 405/14

(52) U.S. Cl. CPC C07D 405/14; C07B 2200/13

(57) ABSTRACT

The present disclosure relates to solid state forms of Entrectinib, processes for the preparation thereof, pharmaceutical formulations/compositions thereof, and methods of use thereof.

### CROSS REFERENCE TO RELATED APPLICATIONS

The present application claims the benefit of and priority to U.S. Provisional Patent Application No. 62/789,577, filed on Jan. 8, 2019, the entire disclosure of which is incorporated herein by reference.

### TECHNICAL FIELD OF THE INVENTION

The present disclosure relates to solid state forms of Entrectinib, processes for the preparation thereof, pharmaceutical formulations/compositions thereof, and methods of use thereof.

### BACKGROUND OF THE INVENTION

Entrectinib, N-(5-[(3,5-difluorophenyl)methyl]-1H-indazol-3-yl)- 4-(4-methylpiperazin-1-yl)-2-Roxan-4-yl)aminoThenzamide, having the following formula:

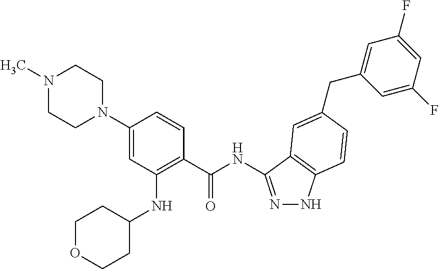 [SMILES: CN1CCN(c2ccc(C(=O)Nc3n[nH]c4ccc(Cc5cc(F)cc(F)c5)cc34)c(NC3CCOCC3)c2)CC1]

is a selective tyrosine kinase inhibitor (TKI), of the tropomyosin receptor kinases (Trk) A, B and C, C-ros oncogene 1 (ROS1) and anaplastic lymphoma kinase (ALK), which is being developed by Ignyta for the treatment of cancer, particularly for the treatment of neuroblastoma and for the treatment of TrkA-, TrkB-, TrkC-, ROS1- and ALK-positive non-small cell lung cancer (NSCLC) and metastatic colorectal cancer (mCRC). Entrectinib was approved in the US for the treatment of ROS1 positive non small cell lung cancer (NSCLC) and for the treatment of solid tumors that have a neurotrophic tyrosine kinase (NTRK) gene fusion. Entrectinib is marketed in the US under the tradename ROZLYTREK™ and is distributed by Genentech.

Entrectinib is described in U.S. Pat. No. 8,299,057. Solid state forms of Entrectinib, including forms 1 and 2 are described in WO2013174876. Further, crystalline form 4 of Entrectinib is disclosed in WO2017/202674.

Polymorphism, the occurrence of different crystal forms, is a property of some molecules and molecular complexes. A single compound, like Entrectinib, may give rise to a variety of polymorphs having distinct crystal structures and physical properties like melting point, thermal behaviors (e.g. measured by thermogravimetric analysis—“TGA”, or differential scanning calorimetry—“DSC”), X-ray powder diffraction (XRPD) pattern, infrared absorption fingerprint, Raman absorption fingerprint, and solid state (¹³C—) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorphic as well as chemical stability) and shelf-life. These variations in the properties of different salts and solid state forms may also provide improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts and solid state forms and solvates of a pharmaceutical product can provide materials having desirable processing properties, such as ease of handling, ease of processing, storage stability, and ease of purification or as desirable intermediate crystal forms that facilitate conversion to other salts or polymorphic forms. New polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for additional salts and solid state forms (including solvated forms) of Entrectinib.

### SUMMARY OF THE INVENTION

The present disclosure relates to solid state forms of Entrectinib, processes for preparation thereof, and pharmaceutical compositions comprising this solid state form.

The present disclosure also provides use of the solid state forms of Entrectinib for preparing other solid state forms of Entrectinib, Entrectinib salts and solid state forms of an Entrectinib salt.

The present disclosure also provides solid state forms of Entrectinib of the present disclosure for uses in the preparation of other solid state forms of Entrectinib, Entrectinib salts and solid state forms of an Entrectinib salt.

In another embodiment, the present disclosure encompasses the described solid state forms of Entrectinib for use in the preparation of pharmaceutical compositions and/or formulations, optionally for the treatment of cancer, preferably selected from the group consisting of neuroblastoma, TrkA-, TrkB-, TrkC-, ROS1- and ALK-positive non-small cell lung cancer (NSCLC) and metastatic colorectal cancer (mCRC) or selected from ROS1 positive non small cell lung cancer (NSCLC) and solid tumors that have a neurotrophic tyrosine kinase (NTRK) gene fusion.

In another embodiment, the present disclosure encompasses uses of the described solid state forms of Entrectinib for the preparation of pharmaceutical compositions and/or formulations.

The present disclosure further provides pharmaceutical compositions comprising one or more solid state forms of Entrectinib according to the present disclosure.

In yet another embodiment, the present disclosure encompasses pharmaceutical formulations comprising one or more of the described solid state forms of Entrectinib and at least one pharmaceutically acceptable excipient.

The present disclosure encompasses processes to prepare said pharmaceutical formulations of Entrectinib comprising combining one or more of the described solid state forms and at least one pharmaceutically acceptable excipient.

The solid state forms defined herein as well as the pharmaceutical compositions or formulations of the solid state forms of Entrectinib can be used as medicaments, particularly for the treatment of cancer, cancer, preferably selected from the group consisting of neuroblastoma, TrkA-, TrkB-, TrkC-, ROS1- and ALK-positive non-small cell lung cancer (NSCLC) and metastatic colorectal cancer (mCRC) or selected from ROS1 positive non small cell lung cancer (NSCLC) and solid tumors that have a neurotrophic tyrosine kinase (NTRK) gene fusion.

The present disclosure also provides methods of treatment of cancer, preferably selected from the group consisting of neuroblastoma, TrkA-, TrkB-, TrkC-, ROS1- and ALK-positive non-small cell lung cancer (NSCLC) and metastatic colorectal cancer (mCRC), comprising administering a therapeutically effective amount of one or more of the solid state forms of Entrectinib of the present disclosure, or at least one of the herein described pharmaceutical compositions or formulations, to a subject suffering from cancer, preferably selected from the group consisting of neuroblastoma, TrkA-, TrkB-, TrkC-, ROS1- and ALK-positive non-small cell lung cancer (NSCLC) and metastatic colorectal cancer (mCRC) or selected from ROS1 positive non small cell lung cancer (NSCLC) and solid tumors that have a neurotrophic tyrosine kinase (NTRK) gene fusion.

The present disclosure also provides uses of the solid state forms of Entrectinib of the present disclosure, or at least one of the above pharmaceutical compositions or formulations for the manufacture of medicaments for treating cancer, preferably selected from the group consisting of neuroblastoma, TrkA-, TrkB-, TrkC-, ROS1- and ALK-positive non-small cell lung cancer (NSCLC) and metastatic colorectal cancer (mCRC) or selected from ROS1 positive non small cell lung cancer (NSCLC) and solid tumors that have a neurotrophic tyrosine kinase (NTRK) gene fusion.

### BRIEF DESCRIPTION OF THE FIGURES

FIG. 1 shows an X-ray powder diffraction (XRPD) pattern of amorphous Entrectinib.

FIG. 2 shows an X-ray powder diffraction (XRPD) pattern of Form C of Entrectinib.

FIG. 3 shows a Differential Scanning calorimetry (DSC) thermogram of Form C of Entrectinib.

FIG. 4 shows a Thermogravimetric analysis (TGA) spectrum of Form C of Entrectinib.

### DETAILED DESCRIPTION OF THE INVENTION

The present disclosure relates to solid state forms of Entrectinib, to processes for preparation thereof and to pharmaceutical compositions and formulations comprising it. The disclosure also relates to the conversion of the solid state forms of Entrectinib of the present disclosure to Entrectinib salt and/or solid state forms thereof

The solid state form of Entrectinib according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents, adhesive tendencies and advantageous processing and handling characteristics such as compressibility, and bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms.

The solid state form of Entrectinib referred to herein as being characterized by graphical data “as depicted in” a Figure will thus be understood to include any Entrectinib, characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, solid states of Entrectinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject solid state form of Entrectinib. In some embodiments of the disclosure, the described solid state forms of Entrectinib may contain from about 1% to about 20% (w/w), from about 5% to about 20% (w/w), or from about 5% to about 10% (w/w) of one or more solid state forms of Entrectinib.

As used herein, unless stated otherwise, XRPD peaks reported herein are preferably measured using CuKα radiation, λ=1.5418 Å, preferably, XRPD peaks reported herein are measured using CuK α radiation, λ=1.5418 Å, at a temperature of 25±3° C.

As used herein, the term “isolated” in reference to solid state forms of Entrectinib of the present disclosure corresponds to solid state form of Entrectinib that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

As used herein, the expression “wet crystalline form” refers to a polymorph that was not dried using any conventional techniques to remove residual solvent. Examples for such conventional techniques can be, but not limited to, evaporation, vacuum drying, oven drying, drying under nitrogen flow, and the like.

As used herein, the expression “dry crystalline form” refers to a polymorph that was dried using any conventional techniques to remove residual solvent. Examples of such conventional techniques can be, but are not limited to, evaporation, vacuum drying, oven drying, drying under nitrogen flow, and the like.

As used herein, and unless stated otherwise, the term “anhydrous” in relation to solid state forms of Entrectinib relates to a crystalline Entrectinib which does not include any crystalline water (or other solvents) in a defined, stoichiometric amount within the crystal. Moreover, an “anhydrous” form does not contain more than about 1% (w/w) of either water or organic solvents as measured for example by TGA.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or a crystallization may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding (methyl tert-butyl ether) MTBE (1.5 v/v) to a 100 ml reaction mixture would indicate that 150 mL of MTBE was added.

As used herein, the term “reduced pressure” refers to a pressure of about 10 mbar to about 500 mbar.

As used herein, and unless indicated otherwise, the term “thermo-dynamical stability” in relation to salts and solid state forms of Entrectinib refers to resistance of the solid state form to polymorphic conversion under certain conditions, for example, heating, melting or dissolving. In some embodiments, the term refers to less than about 20% (w/w), about 10% (w/w), about 5% (w/w), about 1% (w/w), about 0.5% (w/w), or about 0% (w/w) conversion of the crystalline Entrectinib to any other solid state form of Entrectinib as measured by XRPD. In some embodiments, the conversion is about 1% (w/w) to about 20% (w/w), about 1% (w/w) to about 10% (w/w) or about 1% (w/w) to about 5% (w/w).

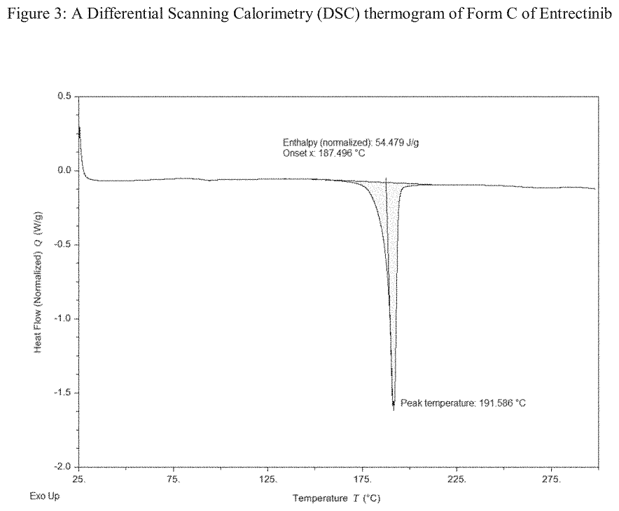

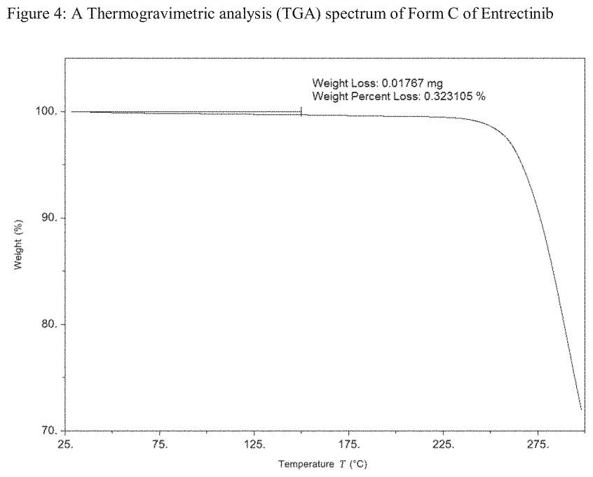

The present disclosure comprises a crystalline form of Entrectinib designated as Form C. The crystalline Form C of Entrectinib can be characterized by data selected from one or more of the following: an XRPD pattern having peaks at 4.6, 7.2, 18.0, 21.6, and 25.2 degrees 2-theta±0.2 degrees 2-theta; an XRPD pattern substantially as depicted in FIG. 2; or combinations of these data. Crystalline Form C of Entrectinib may be further characterized by data selected from one or more of the following: an XRPD pattern having peaks at 4.6, 7.2, 18.0, 21.6, and 25.2 degrees two theta±0.2 degrees two theta; and also having one, two, three, four or five additional peaks selected from 11.6, 12.6, 16.3, 27.6, and 30.8 degrees two theta±0.2 degrees two theta, or selected from 8.1, 11.6, 12.6, 16.3 and 20.1 degrees two theta±0.2 degrees two theta; or combinations of these data. Crystalline Form C may be a solvated or anhydrous form. Anhydrous crystalline Form C of Entrectinib may alternatively or additionally be characterized by data selected from one or more of the following: a DSC thermogram as depicted in FIG. 3; a TGA thermogram as depicted in FIG. 4. Crystalline Form C of Entrectinib shows a melting endothermic peak onset at about 187.5° C. according to DSC thermogram. Further, anhydrous crystalline Form C of Entrectinib shows weight loss of less than 1% of the weight of the sample in a TGA thermogram upon heating up to 150° C. Preferably, Form C shows weight loss of less than 0.5% of the weight of the sample in a TGA thermogram, most preferably Form C shows weight loss of about 0.3% of the weight of the sample in a TGA thermogram upon heating up to 150° C.

Crystalline Form C of Entrectinib may be characterized by each of the above characteristics alone/or by all possible combinations, e.g. by XRPD pattern having peaks at 4.6, 7.2, 18.0, 21.6, and 25.2 deg-2-theta±0.2 deg 2-theta and an XRPD pattern as depicted in FIG. 2.

The present disclosure also provides the use of the solid state forms of Entrectinib of the present disclosure for preparing other solid state forms of Entrectinib and/or solid state forms of Entrectinib salts.

In another embodiment the present disclosure encompasses the use of the above described solid state form of Entrectinib, for the preparation of pharmaceutical compositions and/or formulations.

The present disclosure further provides pharmaceutical compositions comprising the solid state form of Entrectinib described above.

In yet another embodiment, the present disclosure encompasses pharmaceutical formulations comprising the above described solid state form of Entrectinib, and at least one pharmaceutically acceptable excipient.

The present disclosure moreover encompasses processes to prepare said formulations of Entrectinib and solid state forms thereof comprising combining Entrectinib or solid state forms thereof of the present disclosure, and at least one pharmaceutically acceptable excipient.

In another embodiment, the present disclosure encompasses the above described solid state form of Entrectinib, for use in medicine, preferably for the treatment cancer, preferably selected from the group consisting of neuroblastoma, TrkA-, TrkB-, TrkC-, ROS1- and ALK-positive non-small cell lung cancer (NSCLC) and metastatic colorectal cancer (mCRC) or selected from ROS1 positive non small cell lung cancer (NSCLC) and solid tumors that have a neurotrophic tyrosine kinase (NTRK) gene fusion.

The present disclosure also provides methods of treating cancer, comprising administering a therapeutically effective amount of the solid state form of Entrectinib of the present disclosure, or at least one of the above pharmaceutical compositions or formulations, to a subject suffering from cancer, preferably selected from the group consisting of neuroblastoma, TrkA-, TrkB-, TrkC-, ROS1- and ALK-positive non-small cell lung cancer (NSCLC) and metastatic colorectal cancer (mCRC) or selected from ROS1 positive non small cell lung cancer (NSCLC) and solid tumors that have a neurotrophic tyrosine kinase (NTRK) gene fusion. or otherwise in need of the treatment.

The present disclosure also provides the use of the solid state form of Entrectinib of the present disclosure, or at least one of the above pharmaceutical compositions or formulations, for the manufacture of a medicament for treating cancer, preferably selected from the group consisting of neuroblastoma, TrkA-, TrkB-, TrkC-, ROS1- and ALK-positive non-small cell lung cancer (NSCLC) and metastatic colorectal cancer (mCRC) or selected from ROS1 positive non small cell lung cancer (NSCLC) and solid tumors that have a neurotrophic tyrosine kinase (NTRK) gene fusion.

The present disclosure further provides the solid state form of Entrectinib of the present disclosure, or the above pharmaceutical compositions or formulations for use in medicine, especially for treating cancer, preferably selected from the group consisting of neuroblastoma, TrkA-, TrkB-, TrkC-, ROS1- and ALK-positive non-small cell lung cancer (NSCLC) and metastatic colorectal cancer (mCRC) or selected from ROS1 positive non small cell lung cancer (NSCLC) and solid tumors that have a neurotrophic tyrosine kinase (NTRK) gene fusion..

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

### X-ray Powder Diffraction Pattern (“XRPD”) Method:

Sample after being powdered in a mortar and pestle is applied directly on a silicon plate holder. The X-ray powder diffraction pattern was measured with Philips X′Pert PRO X-ray powder diffractometer, equipped with Cu irradiation source=1.54184 Å (Ångstrom), X′Celerator (2.022° 2θ) detector. Scanning parameters: angle range: 3-40 deg., step size 0.0167, time per step 37 s, continuous scan. The described peak positions were determined without using silicon powder as an internal standard in an admixture with the sample measured.

### Differential Scanning Calorimetry (DSC) Method

DSC analysis was performed at instrument Discovery DSC (TA Instruments), at measurement parameters: sample mass—2.2 mg, heating rate—10° C./min, sample pan—aluminum pin hole hermetic, and under nitrogen flow of 50 mL/min.

### Thermogravity Analysis (TGA) Method

TGA analysis was performed on instruments Discovery TGA (TA Instruments), at measurement parameters: sample mass—5.5 mg, heating rate—10° C./min, sample pan—aluminum (open), and under nitrogen flow of 50 mL/min.

Entrectinib crystal Form 1, which is used as the starting material, may be prepared for example according to the processes described in WO 2013/174876 or according to example 6.

### EXAMPLES

### Example 1

### Preparation of Amorphous Entrectinib

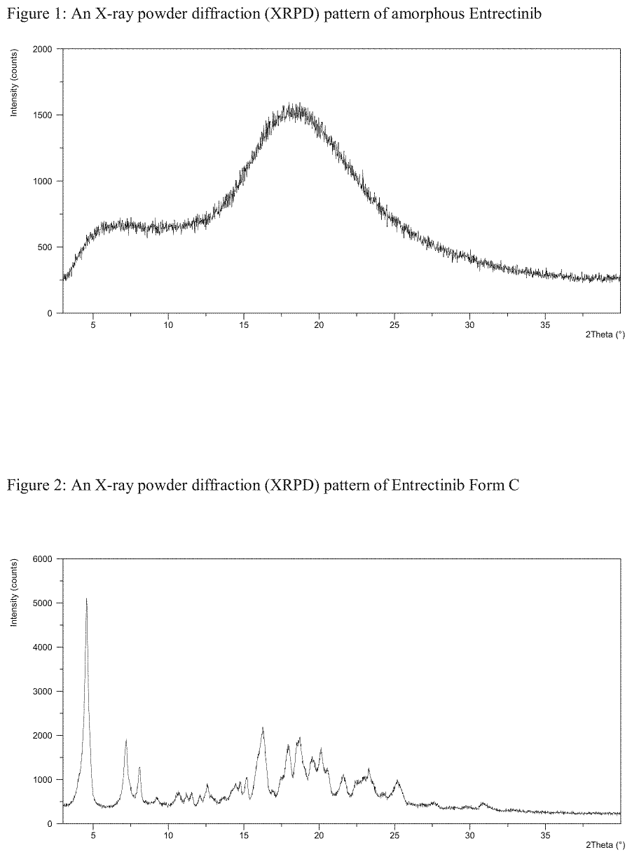

500 mg of Entrectinib crystal form I was subjected to ball mill milling by agate ball mill (2 agate balls ϕ=5 mm, frequency: 30 Hz, time: 60 min). The obtained solid was analyzed by XRPD as depicted in FIG. 1.

### Example 2

### Preparation of Entrectinib Form C, Anhydrous

500 mg of amorphous Entrectinib obtained by Example 1, was exposed for 7 days to vapour of methanol at room temperature, and additional 1 day in open dishes at room conditions (without methanol vapour at 25° C.). The obtained solid was analyzed by XRPD, DSC and TGA as depicted in FIGS. 2, 3 and 4, respectively.

### Example 3

### Preparation of Entrectinib Form C

25 mg of amorphous Entrectinib obtained by Example 1, was exposed for 7 days to vapour of solvent mixture ethanol: water (volume ration 1:1) at room temperature. Obtained solid was analyzed by XRPD, and corresponds to form C.

### Example 4

### Preparation of Entrectinib Form C

25 mg of amorphous Entrectinib obtained by Example 1, was exposed for 7 days to vapour of ethanol at room temperature. Obtained solid was analyzed by XRPD, and corresponds to form C.

### Example 5

### Preparation of Entrectinib Form C

25 mg of amorphous entrectinib obtained by Example 1, was exposed for 7 days to vapour of methanol at room temperature. Obtained solid was analyzed by XRD, and corresponds to form C.

### Example 6

### Preparation of Entrectinib Form 1

140 mg of entrectinib were weighed in a glass vial. 2.0 ml of ethanol were added and the mixture was heated up to 60° C. A clear red solution was obtained. Slowly, 2.0 ml of water were added dropwise in to the solution and a white suspension was obtained. The precipitate was filtered and analyzed by XRPD. Entrectinib Form I was obtained.

1. Crystalline Form C of Entrectinib characterized by an XRPD pattern comprising peaks at 4.6, 7.2, 18.0, 21.6, and 25.2 degrees 2-theta±0.2 degrees 2-theta.

2. Crystalline Form C of Entrectinib according to claim 1, wherein said XRPD pattern further comprises peaks at 8.1, 11.6, 12.6, 16.3 and 20.1 degrees two theta±0.2 degrees two theta.

3. Crystalline Form C of Entrectinib according to claim 1, further characterized by an XRPD pattern according to FIG. 2.

4. Crystalline Form C of Entrectinib according to claim 1, wherein said crystalline Form is unsolvated.

5. A pharmaceutical composition comprising crystalline Form C of Entrectinib according to claim 1.

6. Use of crystalline Form C of Entrectinib according to claim 1 in the preparation of a pharmaceutical composition and/or formulation.

7. A pharmaceutical formulation comprising crystalline Form C of Entrectinib according to claim 1, and at least one pharmaceutically acceptable excipient.

8. Crystalline Form C of Entrectinib according to claim 1 for use as a medicament.

9. Crystalline Form C of Entrectinib according to claim 1, for use in the treatment of cancer, particularly for the treatment of ROS1 positive non small cell lung cancer (NSCLC) and for the treatment of solid tumors that have a neurotrophic tyrosine kinase (NTRK) gene fusion.

10. A method of treating cancer, particularly for the treatment of ROS1 positive non small cell lung cancer (NSCLC) and for the treatment of solid tumors that have a neurotrophic tyrosine kinase (NTRK) gene fusion, comprising administering a therapeutically effective amount of crystalline Form C of Entrectinib according to claim 1, to a subject suffering from said disorder, or otherwise in need of the treatment.
